(19) United States
(12) Patent Application Publication
(10) Pub. No.: US 2025/0302645 A1
(43) Pub. Date: Oct. 2, 2025

(54) POWERED PROSTHETIC FOOT WITH BI-DIRECTIONAL NEURAL INTUITIVE CONTROL AND SENSATION INCLUDING AN IMPROVED CONTROL SYSTEM WITH SENSORIMOTOR FEEDBACK AND CLOSED-LOOP OPERATION WITH MULTIFACETED INPUT/OUTPUT

(71) Applicants: Mark Pitkin, Sharon, MA (US); Hangue Park, Seoul (KR); Boris Prilutsky, Roswell, GA (US); Grigory Raykhtsaum, Sharon, MA (US)

(72) Inventors: Mark Pitkin, Sharon, MA (US); Hangue Park, Seoul (KR); Boris Prilutsky, Roswell, GA (US); Grigory Raykhtsaum, Sharon, MA (US)

(21) Appl. No.: 19/095,697

(22) Filed: Mar. 31, 2025

Related U.S. Application Data
(60) Provisional application No. 63/571,914, filed on Mar. 29, 2024.

(51) Int. Cl. A61F 2/72; A61F 2/46; A61F 2/66; A61F 2/76

(52) U.S. Cl. CPC A61F 2/72; A61F 2002/4632; A61F 2002/6614; A61F 2002/7625; A61F 2002/7635; A61F 2002/7645

(57) ABSTRACT

A powered ankle/foot prosthetic with a closed-loop, neural, bi-directional intuitive control system can be utilized for transtibial amputees with traditional socket prosthetics or for those with osseintegrated prosthetics. Control (efferent) signals from the user to the prosthesis actuator are captured from EMG of residual gastrocnemius and tibialis anterior muscles. Sensory feedback (afferent) information about contact of the prosthetic foot with the ground obtained from a pressure sensor located on the foot bottom is reported to the user's nervous system by electrical stimulation of the residual trans-tibial and sural nerves. Surgically implanted or skin surface electrodes placed on/within the residual ankle flexor and extensor muscles relay recorded EMG to the control electronics mounted on the linear actuator. The control electronics are designed to generate a more natural powered plantarflexion of the ankle joint based on the EMG data, pressure sensor, and other inertial sensors.

### CROSS-REFERENCE TO RELATED APPLICATIONS

This application claims the benefit of U.S. Provisional Patent Application Ser. No. 63/571,914, filed Mar. 29, 2024, the entire contents of which is incorporated herein by reference.

### BACKGROUND OF THE DISCLOSURE

### (1) Field of the Invention

The instant invention generally relates to lower limb prosthetics and powered artificial feet, and more particularly to a novel powered ankle/foot prosthetic with a closed-loop, neural, bi-directional intuitive control system that can be utilized for transtibial amputees with traditional socket prosthetics or for those with osseintegrated prosthetics.

### (2) Description of Related Art

There are about 1.7 million US amputees including former military personnel and war fighters on active duty. Sensory input in lower-limb amputees is critically important for maintaining balance, preventing falls, negotiating uneven terrain and responding to unexpected perturbations.

Although various case studies of control of powered prostheses in individuals with upper and lower limb loss have demonstrated drastic improvements in quality of movements and perception, natural somatosensory feedback from the lost lower limb has not yet been incorporated in current lower limb prosthetics in the market. As an example in the commercial sector of powered ankle-foot prostheses without natural neural control is the Össur Proprio Foot, having an electric actuation at the ankle joint to adjust the ankle angle in swing phase, but being locked during stance, and therefore equivalent to a passive spring foot.

It is important to understand that designs of the existing powered leg prostheses are not “neural” in a sense. That is because the signal from the external environment to the neural system and the signals from the neural systems to the prosthesis' motors are transmitted either via surface electrodes or via the implanted wireless radio gadgets. Both methodologies have low protection from the false signals, which may work for upper limb prostheses, but is unacceptable for lower limb prostheses, where even minor inadequate action of the motors can result in dangerous falls.

To maximize the reliability and adequacy of the neural signal processing for controlling the powered limb prostheses, it was suggested to combine the technologies of powered prosthetics with the technologies of direct skeletal attachment of limb prostheses by placing the wires between the prosthesis and the residuum nerves/muscles in a hollow space inside the skin and bone integrated pylon transcutaneously implanted in the residuum's bone marrow canal.

The first experimental confirmation of such possibility was reported in 2012 with respect to an upper limb prosthetic. An implementation of this approach has been achieved then in the osseointegrated upper limb powered prosthesis attached directly to the amputee's residuum.

Accordingly, an object of the present invention is to provide a lower limb amputee with improved functionality and safety of ambulation compared to the present art. That will be achieved with the osseointegrated prosthesis controlling the powered prosthetic foot with a novel electrical system based on a bi-directional neural closed-loop operation with multifaceted input/output employing intuitive control and sensation.

### SUMMARY OF THE DISCLOSURE

According to exemplary embodiments of the invention, this disclosure presents a human-centered bidirectional lower-limb neuroprosthesis, where the feedback is delivered to the nervous system of the user who controls the active prosthetic joints. This approach is distinct from the concept of machine-centered bidirectional neuroprosthesis, where the feedback is delivered to the microcontroller (machine) controlling the active joints.

Traditional socket prosthetics as well as an osseintegrated prosthetic are described herein. The differences lie mostly in the electronics which interconnect the muscle sensors and nerve stimulators with the control system and the routing of those connections, i.e. surface via wireless connections or with direct leads traveling through the pylon.

The mechanical portions of prosthesis generally comprises a titanium support pylon having a spring dorsiflexion type foot attached at the lower end by an articulating ankle joint. In the socket-type prosthesis the upper end of the pylon is secured to a transtibial socket. In the osseintegrated-type prosthesis, the pylon comprises an elongated a titanium conduit with a porous cladding for safe percutaneous implantation to the residuum with a multichannel cable in its internal conduit for transmitting sensory signals to the nerves and the neural signals to the electrical system.

In some embodiments, the outer surface of the pylon conduit has barbed shape to maximize the surface area of contact with the porous cladding. Further, to maximize the area of osseointegration, the porous cladding of the conduit has variable transversal cross section along the tube length and the outer part of the conduit may have a conical shape with oval cross-section transversal to the longitudinal axis of the conduit.

A powered linear actuator with an integrated current driver extends between a middle portion of the pylon and an extension from the heel of the foot generally where the Achilles tendon would be located.

The control system generally comprises a pressure sensor mounted to the bottom of the spring foot, an accelerometer, an ankle joint angle sensor, soleus and gastrocnemius ElectroMyoGraphic (EMG) sensor electrodes, a 2-channel neural amplifier, low-pass and high-pass filters, tibial and sural nerve stimulation electrodes, a 2-channel neural stimulator, a microcontroller unit (MCU) with serial data interface including SPI and I2C, 4-ch analog-to-digital converter, and wireless transceiver, an antenna on printed circuit board, DC-DC converters to power the overall electrical circuit and the motor, a rechargeable Lithium-polymer battery, a recharging circuitry for the Lithium-polymer battery.

In some embodiments, the EMG and nerve stimulating electrodes may be provided in a separate sensor array with a separate microcontroller, rechargeable battery power supply and wireless transceiver to communicate with the main MCU and motor control.

In some socket-type embodiments, the EMG sensor and nerve stimulating electrodes may be skin surface electrodes communicating wirelessly with the main MCU, or the electrodes may interface with a cable extending from the socket and through the pylon to the main MCU.

In some osseintegrated embodiments, the EMG sensor and nerve stimulating electrodes may also be skin surface electrodes communicating wirelessly with the main MCU, or the electrodes may be surgically implanted and extend through the tibial marrow canal interfacing with a multi-channel cable extending through the pylon to the main MCU.

An external data receiving unit and an external computing system receive operational and feedback data to update the parameters of linear motor control and stimulation based on EMG, pressure sensory output, accelerometer output, ankle joint angle output (real-time), and leg kinematics (offline).

Control (efferent) signals from the user to the prosthesis actuator are captured from EMG of residual ankle plantar and dorsiflexor muscles, specifically the gastrocnemius and tibialis anterior muscles. Sensory feedback (afferent) information about contact of the prosthetic foot with the ground obtained from a pressure sensor located on the foot bottom is reported to the user's nervous system by transcutaneous electrical stimulation of the residual soleus nerve that contains a branch of the tibial nerve with proprioceptive and cutaneous afferents innervating ankle extensors and skin on the plantar surface of the foot. Bipolar surface electrodes placed on the residual ankle flexor and extensor muscles relay recorded EMG to a wireless Bluetooth Low-Energy (BLE) module (mounted on the socket or the prosthesis' pylon) wirelessly sending the data to the control electronics mounted on the linear actuator.

The control electronics are designed to generate plantarflexion of the powered ankle joint based on the EMG data, by controlling a linear actuator to approximate the action of the Achilles tendon. At initial heel strike, the actuator passively extends due to return bias energy of the spring foot. In early mid-stance/stride, the ankle joint is passively dorsiflexed while the actuator is still turned off. In the transition from dorsiflexion to plantarflexion, additional stored energy is released and the actuator actively pulls its arm for plantarflexion. In late stance/stride to end of stride the actuator continues to pull (shorten) to assist plantarflexion and compress the spring-storing energy for the subsequent heel strike and actuator arm extension.

The BLE module delivers sensory feedback to the user via stimulation of the residual tibial nerve, based on the multiple signals from the input sensor. For stimulation of the residual tibial nerve, the bipolar surface electrodes are placed over the residual soleus muscles. The electrode placement is selected for each participant based on perception and thresholds. This stimulation activates groups I, II, and III muscle and cutaneous afferents responsible for tactile and proprioceptive sensations and for evoking spinal locomotor reflexes.

The accelerometer is utilized to classify the status of walking from other leg movements in real time, the pressure sensor measures the gait phase in real time, the angle sensor measures the ankle joint angle, and the motor output torque is measured by the current consumption and controlled with a PWM duty factor.

The microprocessor unit adjusts active duration (phase) of the linear motor, in regards to the gait phase, and the microprocessor unit adjusts stimulation parameters (i.e., amplitude, frequency, and phase of stimulation) to adjust artificial sensory feedback.

The external computing system accumulates the data of motor control parameters and resulting outcome of EMG, sensor, kinematics data, algorithmically evolves motor control parameters (e.g., PWM duty factor, active phase) and delivers them to the built-in electrical system of the prosthesis, based on the history of data and machine learning algorithm.

The external computing system further accumulates the data of stimulation parameters and resulting outcome of EMG, sensor, kinematics data, algorithmically evolves stimulation parameters and delivers them to the built-in electrical system of the prosthesis, based on the history of data and machine learning algorithm.

The result is a novel anthropomorphic lower-limb neuroprosthesis with intuitive bi-directional control and sensation.

While embodiments of the invention have been described as having the features recited, it is understood that various combinations of such features are also encompassed by particular embodiments of the invention and that the scope of the invention is limited by the claims and not the description.

### BRIEF DESCRIPTION OF THE DRAWINGS

While the specification concludes with claims particularly pointing out and distinctly claiming particular embodiments of the instant invention, various embodiments of the invention can be more readily understood and appreciated from the following descriptions of various embodiments of the invention when read in conjunction with the accompanying drawings in which:

FIG. 1 is a schematic illustration of an exemplary embodiment of a socket-type powered prosthetic foot with bi-directional neural intuitive control and sensation (wireless skin surface electrodes) in accordance with the teachings of the present disclosure;

FIG. 2 is a schematic illustration of an exemplary embodiment of a socket-type powered prosthetic foot with bi-directional neural intuitive control and sensation (wired skin surface electrodes) in accordance with the teachings of the present disclosure;

FIG. 3 is a schematic illustration of an exemplary embodiment of an osseintegrated-type powered prosthetic foot with bi-directional neural intuitive control and sensation (wireless skin surface electrodes) in accordance with the teachings of the present disclosure;

FIG. 4 is a schematic illustration of an exemplary embodiment of an osseintegrated-type powered prosthetic foot with bi-directional neural intuitive control and sensation (implanted direct wire electrodes) in accordance with the teachings of the present disclosure;

FIG. 5 is an illustration of an exemplary pylon conduit and porous cladding;

FIG. 5A is an enlarged illustration showing the barbed outer surface of the pylon providing increased contact surface area for enhanced adhesion with the pylon body;

FIG. 6 is a electronic block diagram of the bi-directional neural intuitive control system using a wireless surface skin electrode array (embodiments shown in FIGS. 1 and 3);

FIG. 7 is a electronic block diagram of the bi-directional neural intuitive control system using direct wired electrode connections (embodiments shown in FIGS. 2 and 4;

FIG. 8 graphically illustrates a complete stride cycle of the powered foot wherein (1) at initial heel strike, the actuator passively extends due to return bias energy of the spring foot; (2) in early mid-stance/stride, the ankle joint is passively dorsiflexed while the actuator is still turned off; (3) in the transition from dorsiflexion to plantarflexion, additional stored energy is released and the actuator actively pulls its arm for plantarflexion; and (4) in late stance/stride to end of stride, the actuator continues to pull (shorten) to assist plantarflexion and compress the spring-storing energy for the subsequent heel strike and actuator arm extension; and

FIG. 9 graphically illustrates overall system operation for the bi-directional neural prosthesis with active ankle joint and sensory feedback from the foot wherein EMG recording from soleus and tibialis-anterior muscles controls the linear motor as an active ankle joint and the mapping function between the magnitude of the emulated EMG signal and the duty factor of PWM signal (for motor control) is determined on experimental data (exemplary relationship between the EMG recording from the amputee subjects and the required ankle joint moment); and pressure sensor output from the bottom of the foot is used to stimulate the residual nerve delivering sensory feedback from the foot based on experimental data (exemplary relationship between the pressure sensor output and the pressure perceived by the amputee person).

### DETAILED DESCRIPTION OF THE EXEMPLARY EMBODIMENTS

Certain exemplary embodiments will now be described to provide an overall understanding of the principles of the structure, function, manufacture, and use of the device and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present disclosure. Further, in the present disclosure, like-numbered components of the embodiments generally have similar features, and thus within a particular embodiment each feature of each like-numbered component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Further, to the extent that directional terms like top, bottom, up, or down are used, they are not intended to limit the systems, devices, and methods disclosed herein. A person skilled in the art will recognize that these terms are merely relative to the system and device being discussed and are not universal.

According to exemplary embodiments of the invention, this disclosure presents a human-centered bidirectional lower-limb neuroprosthesis, where the feedback is delivered to the nervous system of the user who controls the active prosthetic joints. This approach is distinct from the concept of machine-centered bidirectional neuroprosthesis, where the feedback is delivered to the microcontroller (machine) controlling the active joints.

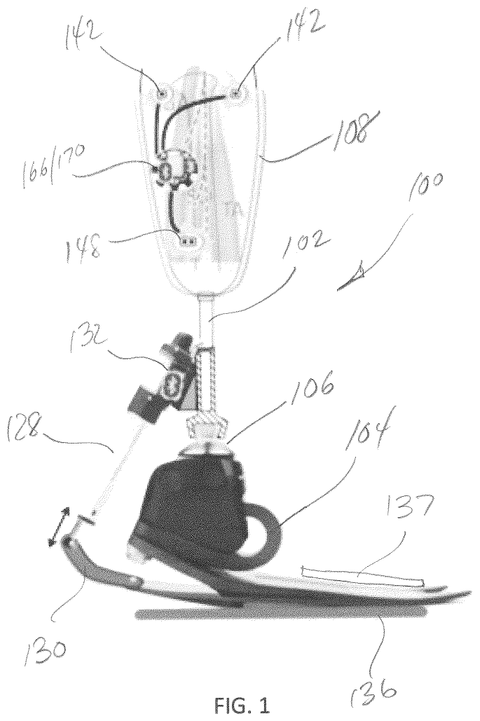

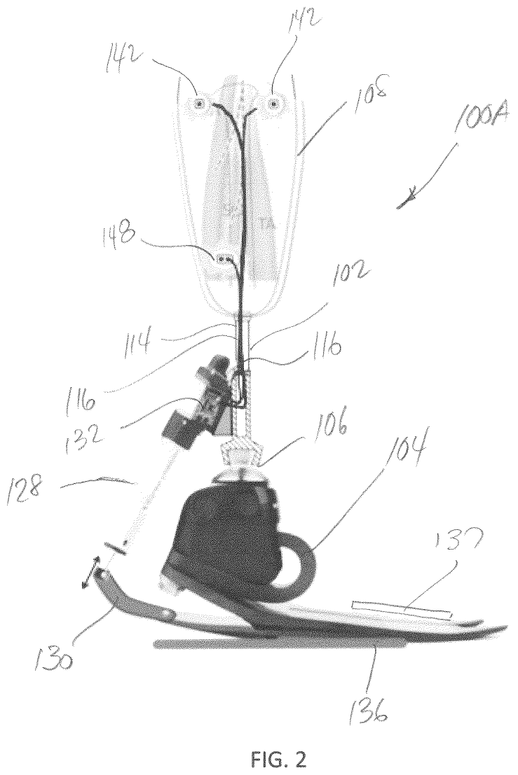

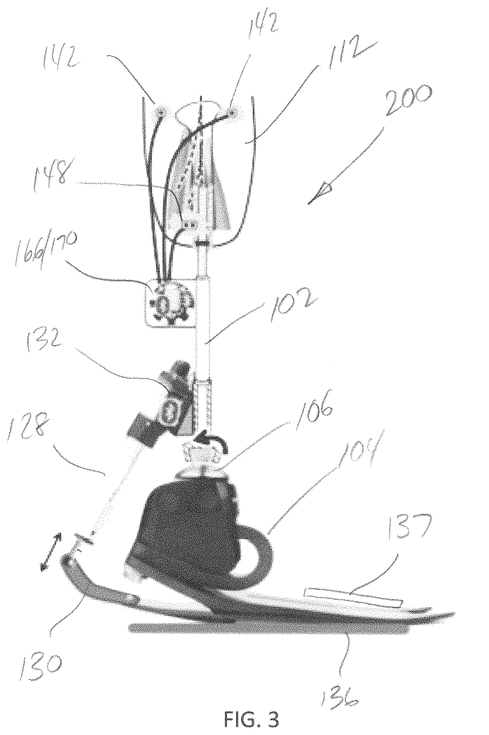

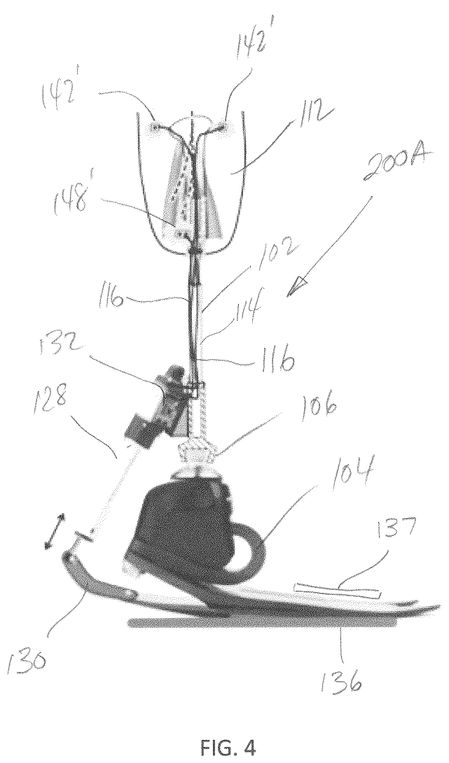

Referring now to FIGS. 1-4, there are four separate embodiments of the invention illustrated, including traditional socket-type prosthetics (FIGS. 1 and 2) as well as osseintegrated-type prosthetics (FIGS. 3 and 4). The differences in these embodiments lie mostly in the electronics which interconnect the muscle sensors and nerve stimulators with the control system, the routing of those connections, i.e. surface via wireless connections or with direct leads traveling through the pylon. Operational control and evolution of the operating parameters of the powered foot is generally the same in all four embodiments.

Illustrated in FIGS. 1 and 2 are socket-type prosthesis 100 and 100A which differ only in the manner of communication between the electronics (Wireless vs. Wired).

Illustrated in FIGS. 3 and 4 are osseointegrated-type prosthesis 200 and 200A which also differ only in the manner of communication between the electronics (Wireless vs. Wired).

Referring to FIGS. 1-4, the common mechanical portions of these prostheses generally comprise a titanium support pylon 102 having a spring dorsiflexion-type passive foot 104 attached at the lower end of the pylon 102 by an articulating ankle joint 106. The illustrated foot 104 is based on the Willowwood™ META™ Flow™ Foot (Willowood, META and Flow are trademarks of Willowwood Global, LLC). However, the invention should not be considered as limited by the exemplary illustrations.

In the socket-type prosthesis 100, 100A the upper end of the pylon 102 is secured to a transtibial socket 108 which is conventional in the art.

In the osseintegrated-type prosthesis 200, 200A, the upper end of the pylon 102 comprises includes a porous titanium cladding 110 for safe implantation to the patient residuum 112.

In some embodiments 100A, 200A (FIGS. 2 and 4), the pylon 102 may include an internal conduit 114 with multichannel cabling 116 in the internal conduit for transmitting sensory electrode signals to the nerves and neural electrode signals to a electrical control system (to be further discussed hereinbelow).

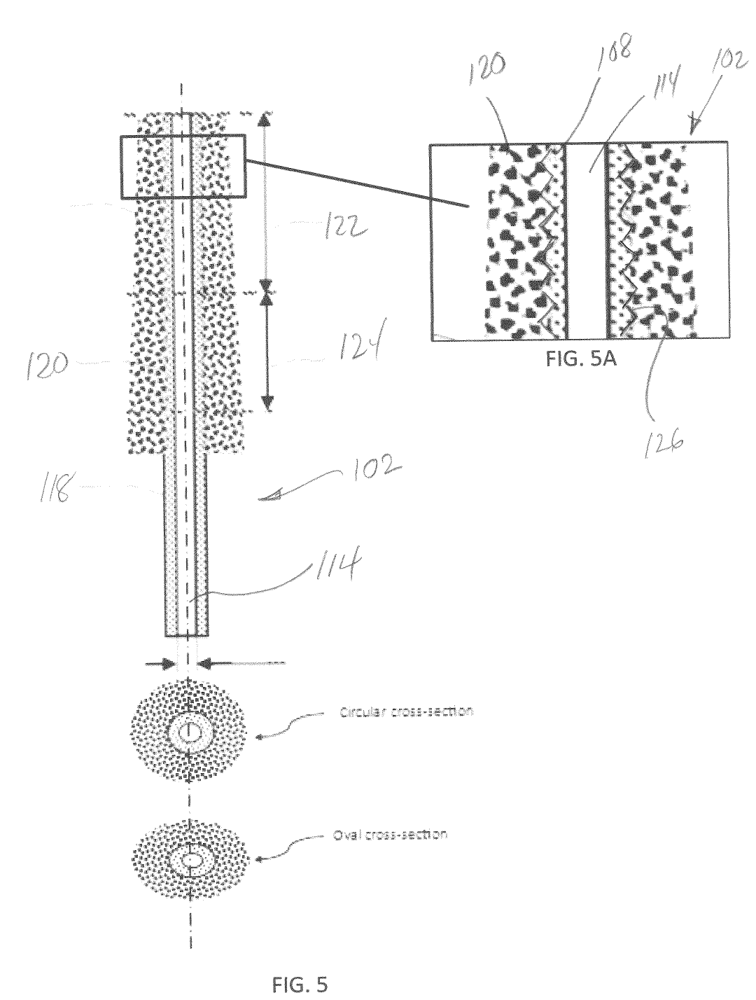

Turning to FIGS. 5 and 5A, in the osseointegrated-type embodiments 200, 200A, the pylon 102 includes a main support portion 118 with the central conduit 114 extending therethrough, and a porous cladding layer 120 on the upper end. The porous cladding layer is defined in two areas comprising a upper portion 122 of the cladding for bone ingrowth and 124 a lower portion of the cladding for skin ingrowth.

Referring briefly to FIG. 5A, the outer surface of the pylon 102 has barbed or grooved surface features 126 to maximize the surface area of contact with the outer porous titanium cladding.

Turning back to the lower portion of FIG. 5, to further maximize the area of osseointegration, the porous cladding 120 of the pylon 102 may have a variable transversal cross section along the tube length and the outer part of the conduit may have a conical shape with oval cross-section transversal to the longitudinal axis of the conduit.

The inner walls of the tube conduit 114 may be coated with submicron layer of pure silver or other antimicrobial coating. The volume fraction of the porous cladding 120 is within 20%-70% of the total conduit volume. The conduit body and cladding 118, 120 may be made with pure or alloyed titanium or other similar biocompatible metal.

Referring back to FIGS. 1-4, a powered linear actuator 128 with an integrated current driver extends between a middle portion of the pylon support 118 and an extension 130 from the heel of the foot generally where the Achilles tendon would be located.

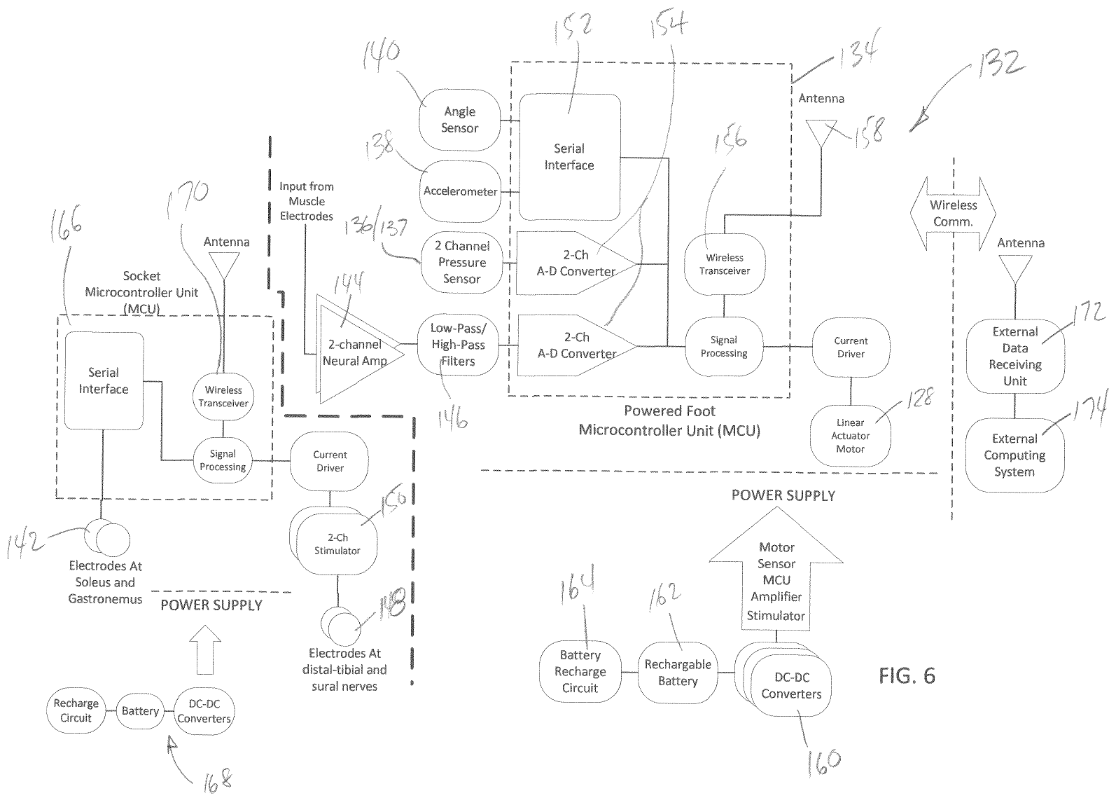

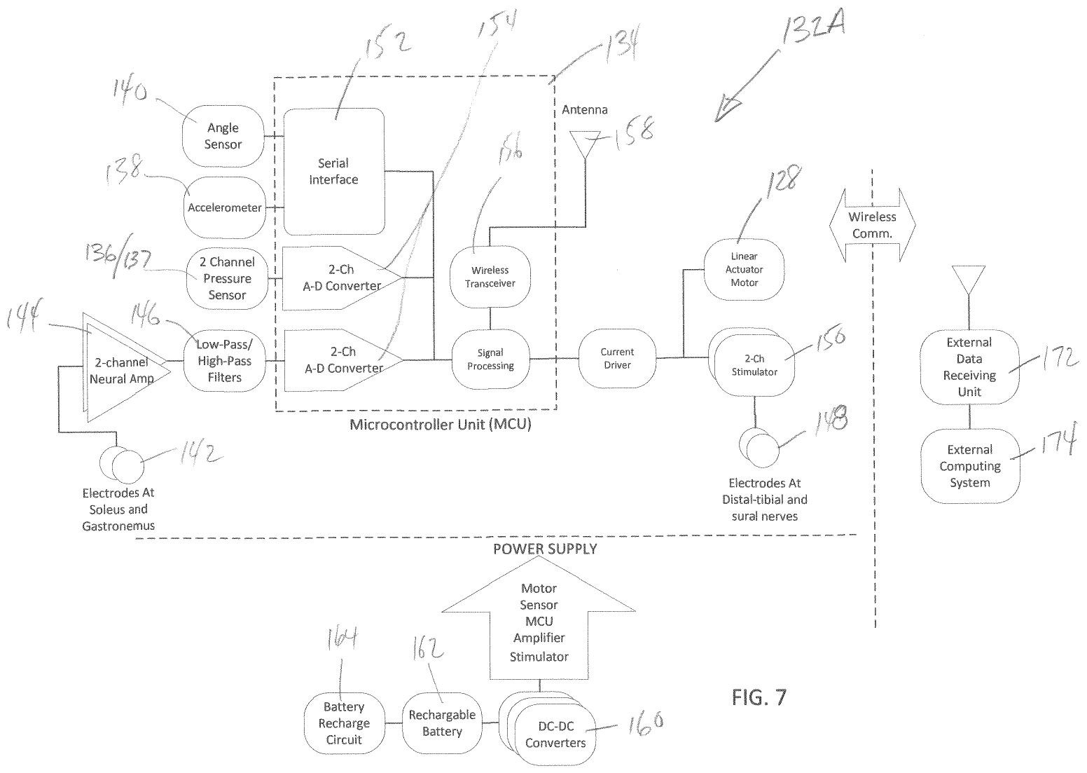

Referring now to FIGS. 6 and 7, there are illustrated two control systems 132, 132A differing only in wireless (132 FIG. 6) and wired (132A FIG. 7) communication between the main controller 132 and the sensory/stimulator electrodes.

The control systems 132 generally comprise a first pressure sensor 136 mounted to the bottom of the spring foot (plantar sensor), a second pressure sensor 137 mounted to the top of the spring foot (dorsal sensor), an accelerometer 138, an ankle joint angle sensor 140, soleus and gastrocnemius ElectroMyoGraphic (EMG) sensor electrodes 142, a 2-channel neural amplifier 144, low-pass and high-pass filters 146, distal-tibial and sural nerve stimulation electrodes 148, a 2-channel neural stimulator 150, microcontroller unit (MCU) 134 with serial data interface 152 including SPI and I2C, 4-ch analog-to-digital converter 154, and wireless transceiver 156, an antenna 158 on printed circuit board, DC-DC converters 160 to power the overall electrical circuit components and the motor 128, a rechargeable Lithium-polymer battery 162, a recharging circuitry 164 for the Lithium-polymer battery.

In some embodiments (FIGS. 1, 3 and 6 (wireless configuration)), the EMG and nerve stimulating electrodes 142, 148 may be provided in a separate sensor array with a separate microcontroller 166, rechargeable battery power supply 168 and wireless transceiver 170 to communicate with the main MCU 134 and motor control.

In some embodiments (FIGS. 1-3), the EMG sensor and nerve stimulating electrodes 142, 148 may be skin surface electrodes communicating wirelessly with the main MCU, or the electrodes 142,148 may interface with cabling 116 extending from the socket 112 and through the pylon 102 to the main MCU 134.

In some osseintegrated embodiments 200A (FIGS. 4 and 7), the EMG sensor and nerve stimulating electrodes 142′,148′ may be surgically implanted and extend through the tibial marrow canal interfacing with cabling 116 extending through the pylon 102 to the main MCU 134.

An external data receiving unit 172 and an external computing system 174 receive operational and feedback data to evolve and update the parameters of linear motor control and stimulation based on EMG, pressure sensory output, accelerometer output, ankle joint angle output (real-time), and leg kinematics (offline) (FIGS. 6 and 7).

Generally, control (efferent) signals from the user to the prosthesis actuator are captured from EMG of residual ankle plantar and dorsiflexor muscles, specifically the gastrocnemius and tibialis anterior muscles (sensor electrodes 142). Sensory feedback (afferent) information about contact of the prosthetic foot with the ground obtained from the pressure sensor 136 located on the foot bottom is reported to the user's nervous system by transcutaneous electrical stimulation of the residual soleus nerve (stimulator electrodes 148). The residual soleus nerve contains a branch of the tibial nerve with proprioceptive and cutaneous afferents innervating ankle extensors and skin on the plantar surface of the foot. In a wireless configuration (FIG. 6) electrodes 142 placed on the residual ankle flexor and extensor muscles relay recorded EMG to a wireless Bluetooth Low-Energy (BLE) module (mounted on the socket or the prosthesis' pylon) wirelessly sending the data to the control electronics mounted on the linear actuator. In a wired configuration (FIG. 7) electrodes 142 placed on the residual ankle flexor and extensor muscles relay recorded EMG directly to the main microcontroller 134.

The stimulator electrodes 148 receive data similarly in each configuration.

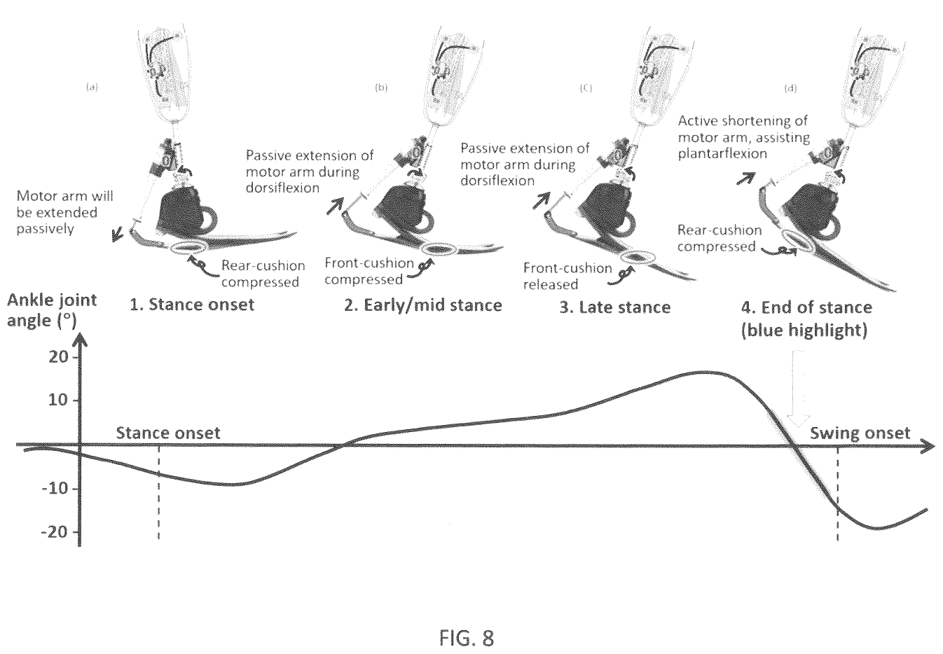

Turning to FIG. 8, the MCU is programmed with a system application which is configured for controlling the system electronics to generate plantarflexion of the powered ankle joint based on the EMG data, by controlling a linear actuator to approximate the action of the Achilles tendon. (1) At heel strike, the actuator 128 passively extends to a neutral position due to return bias energy of the spring foot. (2) In early mid-stance/stride, the ankle joint is dorsiflexed while the actuator 128 is still turned off. (3) In the transition from dorsiflexion to plantarflexion, stored energy in the spring foot is released while the actuator actively pulls its arm for plantarflexion. (4) In late stance/stride to end of stride the actuator continues to pull (shorten) to assist plantarflexion and compress the spring-storing energy for the subsequent heel strike and actuator arm extension.

The control system delivers sensory feedback to the user via stimulation of the residual tibial nerve, based on the multiple signals from the input sensors. For stimulation of the residual tibial nerve, the bipolar surface electrodes are placed over the residual soleus muscles. The electrode placement is selected for each participant based on perception and thresholds. This stimulation activates groups I, II, and III muscle and cutaneous afferents responsible for tactile and proprioceptive sensations and for evoking spinal locomotor reflexes.

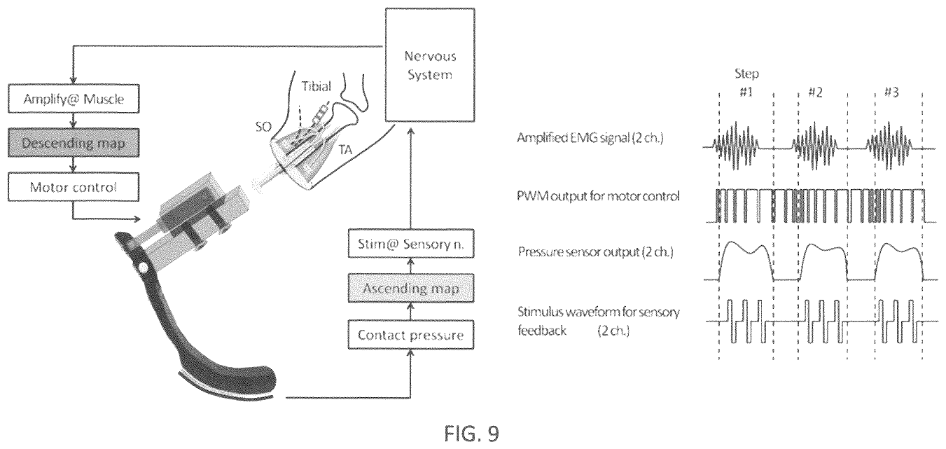

FIG. 9 graphically illustrates overall ascending and descending system operation for the bi-directional neural prosthesis with active ankle joint and sensory feedback from the foot. The EMG recording data from soleus and tibialis-anterior muscles controls the linear motor as an active ankle joint and the mapping function between the magnitude of the emulated EMG signal and the duty factor of PWM signal (for motor control) is determined on experimental data (exemplary relationship between the EMG recording from the amputee subjects and the required ankle joint moment). Pressure sensor output from the bottom of the foot is used to stimulate the residual nerve delivering sensory feedback from the foot based on experimental data (exemplary relationship between the pressure sensor output and the pressure perceived by the amputee person).

An additional dorsal pressure sensor 137 mounted on the top of the spring foot may be utilized to trigger stimulation of the sural nerve evoking a stumbling response when the dorsum of the prosthetic foot contacts an external object.

Key factors to consider for purposes of bi-directional control and operation include the following:

The accelerometer is utilized to classify the status of walking from other leg movements in real time, the pressure sensor measures the gait phase in real time, the angle sensor measures the ankle joint angle, and the motor output torque is measured by the current consumption and controlled with a PWM duty factor.

The microprocessor unit adjusts active duration (phase) of the linear motor, in regards to the gait phase, and the microprocessor unit adjusts stimulation parameters (i.e., amplitude, frequency, and phase of stimulation) to adjust artificial sensory feedback.

The external computing system accumulates the data of motor control parameters and resulting outcome of EMG, sensor, kinematics data, algorithmically evolves motor control parameters (e.g., PWM duty factor, active phase) and delivers them to the built-in electrical system of the prosthesis, based on the history of data and machine learning algorithms running on the external computing system 174.

The external computing system 174 further accumulates the data of stimulation parameters and resulting outcome of EMG, sensor, kinematics data, algorithmically evolves stimulation parameters and delivers them to the built-in electrical system of the prosthesis, based on the history of data and machine learning algorithm.

The control system 132 works as a dynamic closed-loop system with multifaceted real-time measure of various input and outputs (including motor output and neural sensory feedback) to more effectively control the motor output (activation timing and phase duration) with evolving motor control parameters (e.g., PWM duty factor, active phase) and adjust the artificial sensory feedback with an evolving stimulation parameters.
(1) Measured neural signal from the nervous system
   EMG signal onto the ankle extensor/flexor muscles
(2) Measured sensor data at the prosthesis
   Pressure sensor data from the bottom of the prosthesis=>stance/swing phase
   Angle sensor data from the active joint of the prosthesis=>joint angle
   Torque data (motor current) from the active joint of the prosthesis=>ankle loading, push-forward force
   Accelerometer data on the distal-tibia and foot of the prosthesis=>behavioral measure (walking or not)
(3) Measured overall kinematics output
   Full-body kinematics may be gathered from an optical motion capture system
The electrical system will work as a dynamic closed-loop system with multifaceted real-time sensorimotor feedback. This closed loop includes the following adjustments:
(1) Adjust activation timing of the active ankle joint (motor activation)
(2) Adjust torque of the linear motor used for the active ankle joint, using PWM duty factors
(3) Adjust the active duration (phase) of the linear motor, in regards to the gait phase
(4) Adjust artificial sensory feedback (electrical nerve stimulation)
(5) Adjust stimulation parameters (i.e., amplitude, frequency, and phase of stimulation) to adjust artificial sensory feedback

The system further relies on an external data accumulation and processing system 172/174 which accumulates system operational data via wireless link between prosthesis and the external computing system. The external computing system will accumulate the data of motor control parameters and resulting outcome of EMG, sensor, kinematics data and will evolve motor control parameters based on programmed learning algorithms.

Based on the history of data and machine learning algorithm, the external computing system will evolve motor control parameters (e.g., PWM duty factor, active phase) as well as evolve feedback stimulation parameters, and deliver them back to the control system of the prosthesis. Based on these parameters the control system will change motor and control and artificial sensory feedback with the newly evolved stimulation parameters.

The result is a novel anthropomorphic lower-limb neuroprosthesis with intuitive bi-directional control and sensation.

While there is shown and described herein certain specific structures embodying various embodiments of the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. A powered prosthetic foot system comprising:
a support pylon;
a spring dorsiflexion-type passive foot attached at a lower end of the support pylon with an articulating ankle joint;
a powered linear actuator extending between a middle portion of the support pylon and an extension from a heel of the foot;
a closed-loop bi-directional control and neural intuitive feedback system comprising:
a plantar pressure sensor mounted to a bottom of the foot;
an accelerometer;
an ankle joint angle sensor;
ElectroMyoGraphic (EMG) sensor electrodes,
a 2-channel neural amplifier and band pass filters for the EMG sensor electrodes;
nerve stimulation electrodes;
a 2-channel neural stimulator;
a microcontroller unit (MCU) including a serial data interface in communication with said angle sensor and said accelerometer, analog-to-digital converters in communication with said pressure sensor and said EMG electrodes, said MCU further including a processor and current driver in communication with said linear actuator and said neural stimulator, and
a power system to power the control and feedback system and the motor, including respective DC-DC converters, a rechargeable battery and a battery recharging circuit;
said MCU including a programmed system application which is configured for controlling the system electronics and linear actuator to generate plantarflexion of the foot by extension and retraction of the linear actuator based on inputs received from the pressure sensor, the accelerometer, the ankle joint angle sensor, the EMG sensor electrodes,
and further wherein the system application is configured to deliver sensory feedback to the user via said nerve stimulation electrodes based on said inputs.

2. The powered prosthetic foot system of claim 1,
wherein the support pylon pylon includes a main support portion with the central conduit extending therethrough, and a porous cladding layer on the upper end.

3. The powered prosthetic foot system of claim 2,
wherein an a outer surface of the pylon has barbed or grooved surface features to maximize the surface area of contact with the outer porous titanium cladding.

4. The powered prosthetic foot system of claim 2,
wherein the pylon has a variable transversal cross section along a tube length and an outer part of the conduit has a conical shape with oval cross-section transversal to the longitudinal axis of the conduit.

5. The powered prosthetic foot system of claim 2,
wherein inner walls of the central conduit may be coated with submicron layer of pure silver or other antimicrobial coating.

6. The powered prosthetic foot system of claim 2,
wherein a volume fraction of the porous cladding is within 20%-70% of a total conduit volume.

7. The powered prosthetic foot system of claim 2 further comprising:
an external computing system including a wireless transceiver,
said closed-loop bi-directional control and neural intuitive feedback system further comprising a wireless transceiver for communicating with said external computing system,
said external computer system including a system application configured for receiving operational and feedback data, evolving and updating operational parameters of linear actuator control and stimulation based on EMG, pressure sensory output, accelerometer output, ankle joint angle output, and delivering said parameters back to the control and feedback system.

8. The powered prosthetic foot system of claim 1, wherein the accelerometer classifies the status of walking from other leg movements in real time.

9. The powered prosthetic foot system of claim 1 wherein the plantar pressure sensor measures the gait phase in real time.

10. The powered prosthetic foot system of claim 1, wherein the angle sensor measures the ankle joint angle.

11. The powered prosthetic foot system of claim 1, wherein the motor output torque is measured by the current consumption.

12. The powered prosthetic foot system of claim 1 wherein the EMG input is measured from the ankle extensor/flexor muscles (soleus and gastrocnemius).

13. The powered prosthetic foot system of claim 1 wherein neural stimulation feedback is affected through distal-tibial and/or sural nerves.

14. The powered prosthetic foot system of claim 1 wherein the MCU unit adjusts torque used for the active ankle joint, using PWM duty factor.

15. The powered prosthetic foot system of claim 1 wherein the MCU adjusts active duration (phase) of the linear motor, in regards to the gait phase.

16. The powered prosthetic foot system of claim 1 wherein the MCU adjusts stimulation parameters (i.e., amplitude, frequency, and phase of stimulation) to adjust artificial sensory feedback.

17. The powered prosthetic foot system of claim 1 further comprising a dorsal pressure sensor mounted on the top of the foot to trigger stimulation of the sural nerve evoking a stumbling response when the dorsum of the prosthetic foot contacts an external object.
